(19) United States
(12) Patent Application Publication
(10) Pub. No.: US 2015/0359862 A1
(43) Pub. Date: Dec. 17, 2015

(54) Compositions and Methods for Treating Complications Associated with Diabetes

(71) Applicant: BOARD OF REGENTS OF THE UNIVERSITY OF NEBRASKA, Lincoln, NE (US)

(72) Inventor: Keshore R. Bidasee, Omaha, NE (US)

(21) Appl. No.: 14/763,360

(22) PCT Filed: Jan. 30, 2014
(86) PCT No.: PCT/US14/13833

§ 371 Date: Jul. 24, 2015

Related U.S. Application Data
(60) Provisional application No. 61/758,511, filed on Jan. 30, 2013.

(51) Int. Cl. A61K 38/51

(52) U.S. Cl. CPC A61K 38/51; A61K 48/00

(57) ABSTRACT

Compositions and methods for inhibiting diabetes-related complications are provided.

This application claims priority under 35 U.S.C. §119(e) to U.S. Provisional Patent Application No. 61/758,511, filed Jan. 30, 2013. The foregoing application is incorporated by reference herein.

This invention was made with government support under Grant No. R01 HL085061 awarded by the National Institutes of Health. The Government has certain rights in this invention.

### FIELD OF THE INVENTION

The present invention relates to the field of diabetes. Specifically, compositions and methods for inhibiting, treating, and/or preventing diabetes related disorders/complications are disclosed.

### BACKGROUND OF THE INVENTION

Several publications and patent documents are cited throughout the specification in order to describe the state of the art to which this invention pertains. Each of these citations is incorporated herein by reference as though set forth in full.

Varying duration insulins, insulin delivery pumps, glucose monitoring devices, food management, and exercise strategies are available to the more than 1.3 million individuals in the USA with Type 1 diabetes (T1D), to maintain their blood glucose at near physiological levels. This multi-pronged approach has had some success during the last 30 years in reducing the incidences of known cardiovascular complications including blindness, kidney failure, diabetic cardiomyopathy/heart failure, erectile dysfunction and stroke and increasing life expectancy of individuals with T1D by more than 15 years to ±69 years. Unfortunately, the increased longevity is also revealing newer co-morbidities associated with T1D, including cognitive impairment. The latter starts as early as ±10-15 years after the onset of T1D, and manifests as reductions in information processing speed, psychomotor function, cognitive flexibility, visual perception and attention. Interestingly, learning and memory are spared. Recent data from the Diabetes Control and Complications Trial (DCCT) and Epidemiology of Diabetes Interventions and Complications (EDIC) cohorts and other research groups, indicate that the decline in global cognitive function is independent of hypoglycemic episodes but correlates strongly with the degree of microvascular complications. What remains undefined are the intrinsic molecules that initiate cerebral microangiopathy and therapeutic strategies to prevent or slow its development. Currently, there are nine classes of FDA-approved drugs available for global glucose-lowering. However, there are no FDA-approved drugs specifically targeting cardiovascular complications in diabetes. Accordingly, it is evident that new and effective therapeutics for the treatment of complications of diabetes are still needed.

### SUMMARY OF THE INVENTION

In accordance with the present invention, methods for treating, inhibiting, and/or preventing a disease or disorder characterized by overexpression of methylglyoxal in a subject are provided. In a particular embodiment, the methods of the instant invention comprise administering to a subject in thereof a nucleic acid molecule encoding a methylglyoxal degrading enzyme such as glyoxalase-1. In a particular embodiment, the disease or disorder characterized by overexpression of methylglyoxal is diabetes, diabetes related complications, or vascular disease. The nucleic acid molecule encoding a methylglyoxal degrading enzyme may be administered to the subject in a vector, particularly a viral vector. The nucleic acid molecule encoding a methylglyoxal degrading enzyme may be operably linked to an endothelial cell promoter or a smooth muscle cell promoter. In a particular embodiment, the nucleic acid molecule encoding a methylglyoxal degrading enzyme is operably linked to the endothelin-1 promoter. In a particular embodiment, the viral vector is an adeno-associated viral vector.

### BRIEF DESCRIPTIONS OF THE DRAWING

FIG. 1A provides graphs of body weight (top) and blood glucose levels (bottom) in control rats or streptozotocin-induced type-1 diabetic rats (starting weight of ˜220 g) over time after the indicated therapies. FIG. 1B provides graphs of body weight (top) and blood glucose levels (bottom) in control rats or streptozotocin-induced type-1 diabetic rats (starting weight of ˜275 g) over time after the indicated therapies.

FIG. 2A provides a graph of body weight over time for control male and female type 2 diabetic mice (leptin receptor defective) or mice injected with AAV2/9-EndoGlo-1. FIG. 2B provides a graph of blood glucose levels over time for control male and female type 2 diabetic mice (leptin receptor defective) or mice injected with AAV2/9-EndoGlo-1.

FIG. 3 provides a graph of the nesting score for control rats or streptozotocin-induced type-1 diabetic rats after the indicated treatments.

FIGS. 4A and 4B show the percent relaxation of microvessels in streptozotocin-induced type-1 diabetic rats after the indicated treatments. FIG. 4A shows endothelial-dependent relaxation and FIG. 4B shows smooth muscle-dependent relaxation.

FIG. 5A provides images of the cerebral vascular leakage in the cortex, hippocampus, and thalamus of brains of control rats, streptozotocin-induced type-1 diabetic rats, or streptozotocin-induced type-1 diabetic rats treated with AAV2/9 Endo-Glo-1. FIG. 5B provides a graph of the capillary perfusion of the brains of control rats or rats with type 1 diabetes after the indicated treatment.

FIG. 6A provides immunohistochemical images of the endothelium of microvessels of control rats. FIG. 6B provides immunohistochemical images of the endothelium of microvessels of rats with type I diabetes. FIG. 6C provides immunohistochemical images of the endothelium of microvessels of rats with type I diabetes and treated with AAV 2/9-Endo-Glo1.

FIG. 7 provides graphs of the percent cell viability of human brain microvascular endothelial cells (top), human brain microvascular smooth muscle cells (middle), and human astrocytes (bottom) with increasing concentrations of methylglyoxal.

FIG. 8A provides the initial slope of the input-output response and FIG. 8B provides the paired-pulse ratio of synaptic transmissions in hippocampal slices from control rats, rats with type I diabetes, and rats with type I diabetes treated with AAV 2/9-Endo-Glo1.

FIG. 9 provides images of rat brains (top) and a graph of infarct size (bottom) following acute ischemia reperfusion injury. Control rats, rats with type I diabetes, rats with type I diabetes treated with AAV 2/9-Endo-Glo1, and rats with type I diabetes treated with AAV 2/9-Glo1 (lacking the endothelial promoter) were used in the study.

FIG. 10 provides phase images (left column) and images detecting a bovine serum albumin labeled fluorescein isothiocyanate (BAS-FITC) (right column) of kidney sections from control rats (top), rats with type I diabetes (middle), rats with type I diabetes treated with AAV 2/9-Endo-Glo1 (bottom).

### DETAILED DESCRIPTION OF THE INVENTION

Managing cardiovascular complications such as heart failure (cardiomyopathy), kidney failure, retinopathy, and neuropathy associated with diabetes is currently a major health care challenge. Current strategies employ a combination of lifestyle changes (e.g., more exercise and food management), insulins, and other glucose lowering drugs. Lipid lowering and anti-hypertensive drugs are also employed to regulate confounding factors. Unfortunately, the current strategies tend to yield more favorable outcomes with recently diagnosed and younger individuals inflicted with both type 1 and type 2 diabetes than in individuals with chronic and/or long-term diabetes mellitus. Moreover, the level of glycemic control needed to reduce complications is not achievable in all patients due to fear of hypoglycemia, which is more challenging to deal with as symptoms are severe. As the life expectancy for individuals with diabetes continues to increase, additional complications are being revealed including the recently identified decline in cognitive function.

The endothelium is a highly specialized single layer of cells in the lumen of cerebral blood vessels that regulate vascular tone and blood flow by synthesizing and releasing vasodilating (e.g., nitric oxide) and vasoconstricting substances (e.g., thromboxane A). Endothelial cells (ECs) are also an integral component of the blood-brain barrier. Destruction/dysregulation of ECs is an established cause for many cerebral vascular diseases, including Alzheimer's disease and stroke. Endothelial dysfunction is also likely to be the underlying cause for cognitive decline in individuals with T1D. Although hyperglycemia serves as the catalyst, glucose per se is not responsible for causing endothelial dysfunction. The reactive oxidant species and inflammatory mediators generated from shifts in metabolism and cellular biochemistry brought about by hyperglycemia are considered primary candidates.

The microvasculature is a system of small blood vessels within organs that transport nutrients and remove waste. The smallest of these blood vessels are called capillaries. Arterioles and metarterioles transport nutrients to capillaries and venules transport waste from capillaries. Inside the lumen of microvessels is a single layer of specialized cells referred collectively to as the endothelium. These cells synthesize and release into their micro-environments, chemical substances that regulate vascular tone, coagulation and inflammation. The dynamic vascular tone of microvessels which is required for local control of blood flow, blood pressure and nutrient delivery/waste removal within end-organs, is dictated the actions of 3-5 layers of smooth muscle cells (SMC) that respond to the substances secreted by the EC. In T1D, the ability of EC to synthesize and/or secrete these vaso-regulating factors is compromised and this defect may be the initiating cause for end-organ dysfunction. One of the most potent “endothelial-damaging” substances synthesized in the body is methylglyoxal and its production is upregulated in T1D.

Hyperglycemia increases production of two groups of cellular oxidants, reactive oxygen species (ROS) and reactive carbonyl species (RCS). The precursor ROS, superoxide anion O2.⁻, is generated via activation of NAD(P)H oxidases, xanthine oxidases, and complex I and III of the mitochondrial electron transport chain. These species impair endothelium function by reacting with and reducing the primary vasodilating factor, nitric oxide (NO). However, clinical studies employing antioxidant treatments, including vitamin C, vitamin E, and beta-carotene have shown only minimal improvement in cardiovascular functions in individuals with diabetes. One interpretation of these clinical data is that O2.⁻ or its metabolites (OH., HOCl, H₂O₂) are not initiators of cardiovascular dysfunction but rather secondary consequences arising from upstream processes.

Reactive carbonyl species (RCS) are small electrophilic, mono- and di-carbonyl species that include, without limitation, acrolein, N-carboxy(methyl)lysine, N-carboxy(ethyl)lysine 3-deoxyglucosone, glyoxal (GO), imidazolones, 4-hydroxynonenal, Arg-pyrimidine, malondialdehyde, and methylglyoxal (MG). These species are generated from multiple sources, including auto-oxidation of glucose and enzymatic degradation of glucose, lipids, and proteins. RCS are not functionally benign, metabolic by-products; rather, they regulate important cellular and physiologic processes, including cell growth, differentiation, proliferation, apoptosis, and sleep. RCS are not charged and have longer half-lives (minutes to hours) than ROS (msec), allowing them to migrate and exert their effects at locations distant from their generation. The best known effect of RCS is their ability to react with basic amino residues on proteins to form carbonyl adducts, including advanced glycation end-products (AGEs) and advanced lipoxidation end-products (ALEs). RCS can also activate the membrane bound receptors for advanced glycation-end products (RAGEs), triggering production of pro-inflammatory cytokines, chemokines, and adhesion molecules and increased oxidative stress and apoptosis. Cellular levels of RCS are tightly regulated by several RCS-degrading enzymes including glutathione S-transferases (hGSTA4-4 and hGST5.8), aldose reductase, aldehyde dehydrogenase and glyoxalases. In T1D, production of RCS and RCS-degrading enzymes are upregulated. Nevertheless, the amount of RCS produced exceeds the capacity of the RCS-degrading enzymes, resulting in “free RCS” that can irreversibly react with susceptible basic amino acid residues on proteins, altering their functions. Not all RCS that are upregulated in diabetes react with and disrupt the functioning of all cellular proteins. As an example, it was found that although methylglyoxal, glyoxal, malondialdehyde and 4-hydroxynonenal are upregulated in cardiac myocytes, only methylglyoxal and glyoxal form adducts with type 2 ryanodine receptor and sacro(endo)plasmic reticulum Ca²⁺ ATPase in vivo. Methylglyoxal may also be responsible for hyperalgesia seen in patients with diabetes.

Methylglyoxal is the most potent RCS identified to date. Methylglyoxal perturbs intracellular Ca²⁺ homeostasis and impairs mitochondrial function, triggering cell dysregulation and/or death. It is synthesized from oxidation of triose phosphate intermediates, from acetone/acetol via acetone monooxygenase/acetol monooxygenase (AMO), and from proteins via the membrane bound enzyme vascular adhesion protein-1 (VAP-1) and its soluble form serum semicarbazide-sensitive amine oxidase (SSAO). MG is degraded by the glyoxalase system, which consists of two enzymes glyoxalase I (Glo-1) and glyoxalase II (Glo-2) in the presence of glutathione. Methglyoxal may also be degraded by aldose reductase. Glyoxal (GO) is formed by lipid peroxidation and the fragmentation of glycated proteins, and is degraded by the glyoxalase enzymes. Exposure of cardiac myocytes to MG increased cytoplasmic and mitochondria Ca²⁺, and mitochondria superoxide O2.⁻ generation. These data explain the lack of efficacy of anti-oxidant therapy in clinical studies, i.e., they are not the primary species involved in the disease pathogenesis.

The armamentarium of insulins available can reduce the incidence of known cardiovascular complications in individuals with T1D, including blindness, kidney failure, diabetic cardiomyopathy, erectile dysfunction, and stroke and it increases longevity. However, the increased longevity is revealing newer co-morbidities not previously observed, in particular, cognitive decline. To date the mechanisms underlying cognitive decline in individuals with T1D remain unknown and therapeutic strategies to prevent its development are unavailable. The data provided herein identify MG, whose production is upregulated early in T1D, as a causative agent for the cognitive decline, by impairing cerebral vascular reactivity. Smooth muscle cells of the vascular may produce the high concentrations of methylglyoxal in T1D that is negatively impacting the function of endothelial cells. The data also identify Glo-1 overexpression as a novel therapeutic approach for blunting cerebral vascular dysfunction and cognitive impairment in T1D by lowering MG and GO (another substrate) levels. Since microvascular dysfunction is an established cause for other diabetic complications listed above, this novel strategy to lower MG and GO levels, will have therapeutic benefits beyond improving cerebral vascular and cognitive functions in T1D.

In accordance with the instant invention, methods of inhibiting, treating, and/or preventing a disease or disorder characterized by aberrant methylglyoxal levels (e.g., overexpressed) are provided. The instant invention encompasses methods of inhibiting, treating, and/or preventing diabetes (type 1 and/or type 2) and/or diabetes-related complications. The instant invention also encompasses methods of inhibiting, treating, and/or preventing vascular diseases and/or cardiovascular diseases. Examples of diabetes-related complications include, but are not limited to: organ dysfunction (e.g., end organ dysfunction such as of the heart, kidney, eye, foot, and brain), vascular diseases, cardiovascular diseases, heart failure, arterial atherogenesis, renal failure, retinopathy, neuropathy and cognitive impairment (see, e.g., www.diabetes.org/living-with-diabetes/complications/and www.idf.org/complications-diabetes). Examples of end organ complications/dysfunction include without limitation: retinopathy, kidney failure, heart failure, sexual dysfunction, periodontal diseases, and stroke. Examples of cognitive impairments include, without limitation, impairments in psychomotor function, visuo-construction, information processing disease, mental flexibility, and working memory. Examples of vascular diseases include, but are not limited to: cardiovascular disease, heart disease, cardiomyopathy, atherosclerosis, stroke, hypertension, and peripheral arterial disease. Examples of diseases where methylglyoxal has been implicated (e.g., aberrant methylglyoxal levels (e.g., overexpressed)) include, but are not limited to: neurodegeneration, cirrhosis, arthritis, and aging. In a particular embodiment, the methods of the instant invention inhibit, treat, and/or prevent the decrease in cardiac contractility and minimize the increase in blood brain barrier permeability induced by hyperglycemia. In a particular embodiment, the methods of the instant invention improve functions of the heart, kidney, and/or brain in individuals suffering from hyperglycemia and diseases with similar cardiovascular complications. In another embodiment, the methods of the instant invention reduce cerebral infarct size.

The methods of the instant invention comprise administering to a subject a nucleic acid molecule encoding a methylglyoxal degrading enzyme (e.g., glyoxalases (e.g., glyoxalase-1), aldose reductase, aldehyde dehydrogenase (e.g., aldehyde dehydrogenase-9), and 2-oxoaldehyde dehydrogenase) to a subject in need thereof. In a particular embodiment, the methylglyoxal degrading enzyme is glyoxalase-1. In a particular embodiment, the glyoxalase-1 is human glyoxylase-1. Human glyoxalase-1 is described in GenBank GeneID: 2739. GenBank Accession Nos. NM_{—}006708 and NP_{—}006699 provide amino acid and nucleotide sequences of human glyoxalase-1. Compositions comprising a nucleic acid molecule encoding glyoxalase-1 (e.g., a vector comprising the nucleic acid molecule encoding glyoxalase-1) and at least one pharmaceutically acceptable carrier are also encompassed by the instant invention.

In a particular embodiment, the subject is administered a vector comprising the nucleic acid molecule encoding a methylglyoxal degrading enzyme (e.g., glyoxalase-1). In a particular embodiment, the nucleic acid molecule encoding a methylglyoxal degrading enzyme (e.g., glyoxalase-1) is under the control of an endothelial and/or smooth muscle cell promoter. A cell type or tissue specific promoter is a promoter which has greater activity (e.g., at least 2-fold, 3-fold, 4-fold, 5-fold, 10-fold or more) in the desired cell type(s) or tissue(s) compared to other cell types or tissues and/or a promoter that expresses a linked nucleic acid sequence predominantly in the desired cell type(s) or tissue(s) to the general (substantial) or complete exclusion of other cell types or tissues. Examples of endothelial cell promoters include, without limitation, tie1 promoter, tie2/tek promoter, Et-1 promoter, von Willebrand factor promoter, intercellular adhesion molecule 2 (ICAM-2) promoter, endoglin promoter, ICAM-1 promoter, VCAM-1 promoter, Flt-1 promoter, kdr/flk-1 promoter, and endothelin-1 promoter (e.g., the preproendothelin-1 (PPE-1) promoter) (see also U.S. Pat. Nos. 5,888,765; 6,103,527; 6,200,751; 7,067,649; 7,579,327). In a particular embodiment, the endothelial cell promoter is endothelin-1 promoter (e.g., the human endothelin-1 promoter or pre-proendothelin promoter). Lee et al. (J. Biol. Chem. (1990) 10446-50) and Stow et al. (FASEB J. (2011) 25:16-28) provide a nucleotide sequence of the endothelin-1 promoter. Notably, endothelin-1 is synthesized predominantly by vascular endothelial cells and these cells are either removed in number or dysfunctional in diabetes. The promoter was selected, however, because endothelin-1 expression is also upregulated in smooth muscle, macrophages, myocytes and proximal tubule cells in diabetes. Accordingly, although characterized as an endothelial cell promoter, the endothelin-1 is expressed in a few other cell types. Without being bound by theory, this specific upregulation could help target Glo-1 expression in key organs where methylglyoxal has been implicated in the pathophysiology while reducing Glo-1 expression in healthy cells, where glyoxalase-1 overexpression if not needed. In a particular embodiment, the nucleic acid molecule encoding a methylglyoxal degrading enzyme (e.g., glyoxalase-1) is under the control of a smooth muscle cell promoter. Examples of smooth muscle cell promoters include, without limitation, smooth muscle alpha-actin promoter, smooth muscle myosin heavy chain promoter, FRNK promoter, CRP 1 promoter, and SM-22 promoter (e.g., SM-22α promoter (transgelin)). In a particular embodiment, the nucleic acid molecule encoding a methylglyoxal degrading enzyme (e.g., glyoxalase-1) is under the control of a kidney promoter (e.g., a glomeruli promoter or proximal tubules promoter; e.g., to treat kidney dysfunction/failure/disease). Examples of kidney promoters include, without limitation, nephrin promoter (targets glomeruli of kidney), podocin promoter (targets glomeruli of kidney), and Na+/glucose cotransporter (SGLT2) promoter (targets proximal tubules in kidneys). In a particular embodiment, the promoter is not the cytomegalovirus (CMV) immediate early promoter.

The vector of the instant invention may be a plasmid or a viral vector. Viral vectors include, without limitation, adenoviral vectors, adeno-associated virus—(AAV) vectors, and retroviral vectors. The vector may be used to target Glo-1 expression to specific cell types or to key organs where methylglyoxal has been implicated in the pathophysiology of the disease or disorder to be treated. In a particular embodiment, the vector may be used to target Glo-1 expression to endothelial cells and/or smooth muscle cells. For example, a viral vector capable of transducing the desired cell type (e.g., endothelial cells and/or smooth muscle cells) is utilized in the methods. In a particular embodiment, the viral vector is an adeno-associated viral vector. Typically, the genome of the adeno-associated viral vector generally comprises a 5′ adeno-associated virus inverted terminal repeat, a coding sequence (e.g., transgene) operatively linked to a promoter, and a 3′ adeno-associated virus inverted terminal repeat. The adeno-associated viral vector may further comprise additional sequences (e.g., from an adenovirus), which assist in packaging the adeno-associated viral vector into virus particles. The adeno-associated viral vector may be of any serotype. The adeno-associated viral vector may be of a serotype selected from the group consisting of AAV1, AAV2, AAV3, AAV4, AAV5, AAV6, AAV7, AAV8, AAV9, AAV10, AAV11, AAV12, and hybrids thereof (e.g., a combinatorial hybrid of 2, 3, 4, 5, or more serotypes). The adeno-associated viral vector may be a hybrid AAV vectors having a capsid protein (e.g., any one of AAV serotypes 1-12) and genome (e.g., AAV serotype 2) from different AAV. In a particular embodiment, the adeno-associated viral vector is AAV2/9 or AAV 2/1. Methods of synthesizing and preparing adeno-associated viral vectors are well known in the art.

The methods of the instant invention may further comprise the administration of at least one other therapeutic for the treatment, inhibition, and/or prevention of the disease or disorder. The other therapeutic may be administered sequentially and/or simultaneously with the glyoxalase-1 of the instant invention. For example, the nucleic acid molecule of the instant invention may be administered with at least one other drug for the treatment of methylglyoxal-induced vascular dysregulation and/or cardiovascular complications. In a particular example, the methods of the instant invention further comprise administering insulin and/or a blood glucose lowering drug. The insulin may be administered parenteraly and/or pulmonarily. Examples of blood glucose lowering drugs include, without limitation, sulfonylureas (e.g., acetohexamide, chlorpropamide, tolbutamide, glipizide, glyburide), biguanides (e.g., metformin, phenformin, buformin, benfosformin, etoformin, tiformin, proguanil), alpha-glycosidase inhibitors, thiazolidinediones (e.g., glitazones, troglitazone, rosiglitazone, pioglitazone), glinides, meglitinides, GLP analogs, amylin analogs, D-phenylalanine derivatives, DPP-IV inhibitors, bile acid sequestrants, and renal sodium glucose co-transporter inhibitors (e.g., dapaglifozin).

While the instant invention has been described hereinabove through the administration of nucleic acid molecules encoding a methylglyoxal degrading enzyme such as glyoxalase-1, the instant invention also encompasses the administration of a methylglyoxal degrading enzyme as a protein. While the delivery of a nucleic acid molecule has advantages such as increased and prolonged expression, the enzyme may be delivered to the cells of the subject for the therapeutic purposes described herein. For example, PEGylation, nanoparticles (e.g., those comprising biodegradable polymers such as poly lactic acid, polycaprolactone, poly(lactic-co-glycolic acid), chitosan, and/or polyethylene glycol), liposomes, PEGylated liposomes, and receptor-mediated delivery systems may be used to deliver the protein to the cells of a subject. Compositions comprising a glyoxalase-1 and at least one pharmaceutically acceptable carrier are also encompassed by the instant invention.

The compositions of the present invention can be administered by any suitable route, for example, by injection (e.g., for local, direct, or systemic administration), oral, pulmonary, topical, nasal or other modes of administration. The composition may be administered by any suitable means, including parenteral, intramuscular, intravenous, intraarterial, intraperitoneal, subcutaneous, topical, inhalatory, transdermal, intrapulmonary, intraareterial, intrarectal, intramuscular, and intranasal administration. In a particular embodiment, the composition is administered intravenously. In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., Tween 80, Polysorbate 80), anti oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention (see, e.g., Remington's Pharmaceutical Sciences and Remington: The Science and Practice of Pharmacy). The pharmaceutical composition of the present invention can also be prepared, for example, in liquid form, or can be in dried powder form (e.g., lyophilized for later reconstitution).

The therapeutic agents described herein (e.g., nucleic acid molecule encoding a methylglyoxal degrading enzyme such as glyoxalase-1) will generally be administered to a patient as a pharmaceutical preparation. The term “patient” as used herein refers to human or animal subjects. The compositions of the instant invention may be employed therapeutically or prophylactically, under the guidance of a physician.

The compositions comprising the agent of the instant invention may be conveniently formulated for administration with any pharmaceutically acceptable carrier(s). The concentration of agent in the chosen medium may be varied and the medium may be chosen based on the desired route of administration of the pharmaceutical preparation. Except insofar as any conventional media or agent is incompatible with the agent to be administered, its use in the pharmaceutical preparation is contemplated.

The dose and dosage regimen (e.g., titer with regard to viral vectors) of the agent according to the invention that is suitable for administration to a particular patient may be determined by a physician considering the patient's age, sex, weight, general medical condition, and the specific condition for which the agent is being administered to be treated or prevented and the severity thereof. The physician may also take into account the route of administration, the pharmaceutical carrier, and the agent's biological activity. Selection of a suitable pharmaceutical preparation will also depend upon the mode of administration chosen.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment or prevention therapy. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation or prevention of a particular condition may be determined by dosage concentration curve calculations, as known in the art.

The pharmaceutical preparation comprising the agent may be administered at appropriate intervals until the pathological symptoms are reduced or alleviated, after which the dosage may be reduced to a maintenance level. The appropriate interval in a particular case would normally depend on the condition of the patient.

Toxicity and efficacy (e.g., therapeutic, preventative) of the particular formulas described herein can be determined by standard pharmaceutical procedures such as, without limitation, in vitro, in cell cultures, ex vivo, or on experimental animals. The data obtained from these studies can be used in formulating a range of dosage for use in human. The dosage may vary depending upon form and route of administration. Dosage amount and interval may be adjusted individually to levels of the active ingredient which are sufficient to deliver a therapeutically or prophylactically effective amount.

### DEFINITIONS

The following definitions are provided to facilitate an understanding of the present invention:

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions may be employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., diabetes-related complication) resulting in a decrease in the probability that the subject will develop the condition.

A “therapeutically effective amount” of a compound or a pharmaceutical composition refers to an amount effective to prevent, inhibit, or treat a particular disorder or disease and/or the symptoms thereof. For example, “therapeutically effective amount” may refer to an amount sufficient to modulate diabetes-related complications in a subject.

As used herein, the term “subject” refers to an animal, particularly a mammal, particularly a human.

The term “promoter” as used herein refers to a DNA sequence which directs transcription of a polynucleotide sequence operatively linked thereto (e.g., in a cell). The promoter may also comprise enhancer elements which stimulate transcription from the linked promoter. The term “enhancer” refers to a DNA sequence which binds to the transcription initiation complex and facilitates the initiation of transcription at the associated promoter.

A “vector” is a nucleic acid molecule such as a plasmid, cosmid, bacmid, phage, or virus, to which another genetic sequence or element (either DNA or RNA) may be attached/inserted so as to bring about the replication and/or expression of the sequence or element (e.g., under the control of a promoter contained within the vector).

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. An isolated nucleic acid (either DNA or RNA) may further represent a molecule produced directly by biological or synthetic means and separated from other components present during its production.

The following example provides illustrative methods of practicing the instant invention and is not intended to limit the scope of the invention in any way.

### EXAMPLE

### Introduction

Individuals with diabetes mellitus (DM) are living longer thanks to an armamentarium of glucose-lowering drugs, glucose-monitoring devices, food management, and exercise strategies. Unfortunately, they are also continuing to develop cardiovascular diseases and multiple end organ complications including retinopathy, kidney failure, heart failure, sexual dysfunction, periodontal diseases, and stroke at rates 3-4 times higher than the general population (Guariguata et al. (2011) Diabetes Res. Clin. Prac., 94:322-332), co-morbidities that negatively impact daily living activities. In addition to these well known co-morbidities, recent studies have also uncovered slowing in information process speed and psychomotor functions, impaired visuo-construction, attention deficits, impaired mental flexibility, and working memory, and cognitive deficits (Kodl et al. (2008) Endocrine Rev., 29:494-511). These cognitive deficits are independent of hypoglycemic episodes and negatively affect daily living activities. To date, the molecular cues responsible for end-organ complications in T1D remain incompletely defined and specific pharmacological treatments to prevent them are not available.

Clinical studies have firmly established strong correlations between impairment in end-organ function and microvascular defects (Biessels et al. (2008) Lancet Neurol., 7:184-190; Liew et al. (2009) J. Amer. Geriatr. Soc., 57:1892-1896). The reactive di-carbonyl species methylglyoxal (MG) is the leading molecule candidate responsible for endothelial-mediated dysregulation of the microvasculature (Takahashi et al. (2008) Yakugaku zasshi: J. Pharm. Soc. Japan, 128:1443-1448; Dhar et al. (2010) Brit. J. Pharmacol., 161:1843-1856; Brouwers et al. (2010) Diabetologia 53:989-1000). However, the source of the microvascular damaging methylglyoxal is unknown. Because this small-molecule electrophile regulates key physiological functions, including cell differentiation, cell death, and sleep (Hovatta et al. (2005) Nature 438:662-666; Jakubcakova et al. (2013) J. Psychopharmacol., 27:1070-5), globally reducing MG level to minimize microvascular complications in T1D is likely to precipitate undesirable adverse effects. However, identifying and lowering “microvasculature-damaging pools” of MG would be therapeutically effective for reducing end organ-complications in T1D without triggering deleterious side effects.

Rodents globally overexpressing Glo-1 are less likely to develop end-organ complications after induction of T1D and knocking out/down Glo-1 leads to diabetes-like, end-organ complications. Moreover, mice overexpressing the MG-generating enzyme semicarbazide-sensitive amine oxidase (SSAO) also display diabetes-like symptoms. Because MG regulates key cellular and physiological functions including cell differentiation, cell death, and sleep, globally reducing its level to minimize endothelial dysfunction will precipitate an array of undesirable side effects, including anxiety, cancers and irritability. Increasing expression of Glo-1 in SMC prevents endothelial-mediated dysfunction in pial arterioles (a prototype microvessel), the development of diabetic heart failure and the increased water consumption and urination (polydipsia/polyuria), characteristic of T1D. These animals exhibited normal nest construction capability, a daily living activity that requires cognitive integrity including attention, decision-making, visuo-construction, and motor skills. T1D rats overexpressing Glo-1 in SMC did not exhibit signs of pain, distress or abnormal grooming over a nine-week study period and on sacrifice, there were also no visible signs of abnormalities in internal organs. Overexpressing Glo-1 in SMC did not affect SMC-mediated microvessel function. The novel gene-transfer construct created herein, AAV2/9-Glo-1 driven by the endothelin-1 promoter (AAV2/9-Endo-Glo-1), also prevented impairment in synaptic transmission and brain inflammation. Injection of AAV2/9-Endo-Glo-1 one week after the onset of T1D did not lower plasma MG levels, indicating the absence of a cause-effect relationship between plasma MG and microvascular defects. Notably, the amount of free MG in plasma/sera of patients with T1D (<2 μM) is insufficient to cause endothelial cell dysfunction in vitro (>>25 μM). Because of the proximity of EC to SMC in microvessels, EC are especially susceptible to the localized, high concentrations of MG synthesized and released by SMC. Mechanistically, MG perturbs sarcoplasmic reticular Ca²⁺ homeostasis in EC cells (within seconds), increases superoxide (O2.) production and dysregulates mitochondria (10-20 min) and decreases expression of tight junction proteins that hold EC together (within hr).

The findings presented herein indicate that T1D causes SMC to synthesize and secrete sufficient MG to create a microenvironment containing a high concentration of this potent RCS, which ultimately exerts a deleterious functional impact on neighboring EC. This scenario expands on the established concept that in healthy subjects, microvascular EC synthesize and secrete substances that regulate the activities of SMC to control the vascular tone of microvessels. The findings in rats also establishes proof-of-concept that selectively lowering MG in SMC is an effective therapeutic strategy for reducing end organ-complications in T1D with minimal side effects.

### Results

Overexpressing the MG-degrading enzyme glyoxalase-1 (Glo-1) in smooth muscle cells (SMC) using an adeno-associated viral (AAV) strategy prevented endothelium-mediated dysfunction of microvessels, blunted the development of a diabetic cardiomyopathy, polydipsia/polyuria, and minimized impairment in nest construction, a daily living function that requires long-term attention, decision-making, visuo-construction, and motor skills. Surprisingly, the AAV construct that uncovered these provocative findings, AAV2/9-Glo-1 driven by the endothelin-1 promoter, did not lower plasma MG levels in T1D rats, indicating that elevation in plasma levels of MG is unlikely to be the primary MG source responsible for microvascular damage in T1D. These new observations indicate that SMC are a primary source of endothelial-damaging MG in the microvasculature and that lowering this defined sub-pool of MG has significant therapeutic benefits. These new data also explain why the amount of free MG found in plasma/sera of patients with T1D (high nM/low μM) is ˜50× less than that which is required to impair endothelial function in vitro.

The effects of AAV2/9-Endo-Glo-1 on body weight and blood glucose levels in the streptozotocin (STZ)-induced rat model of Type 1 diabetes were assessed. Briefly, the vector was generated by cloning rat Glo-1 into pZac2.1 using Nhe I and Xho I. The endothelin-1 promoter was inserted prior to the Glo-1 encoding sequence in pZac2.1. pAdDelta A6 and pAAV2/9 were subsequently used to create AAV2/9-Endo-Glo1.

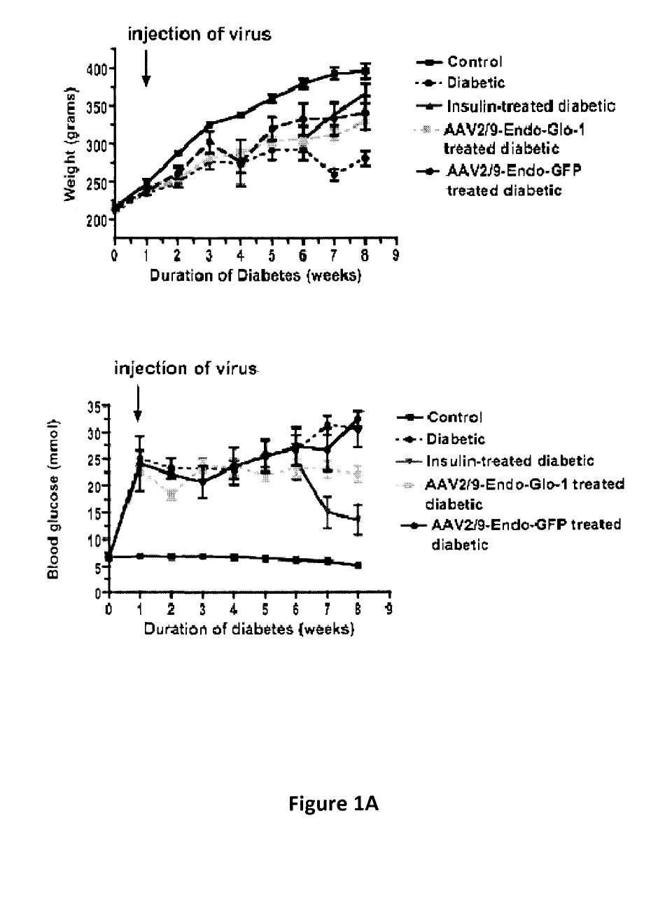

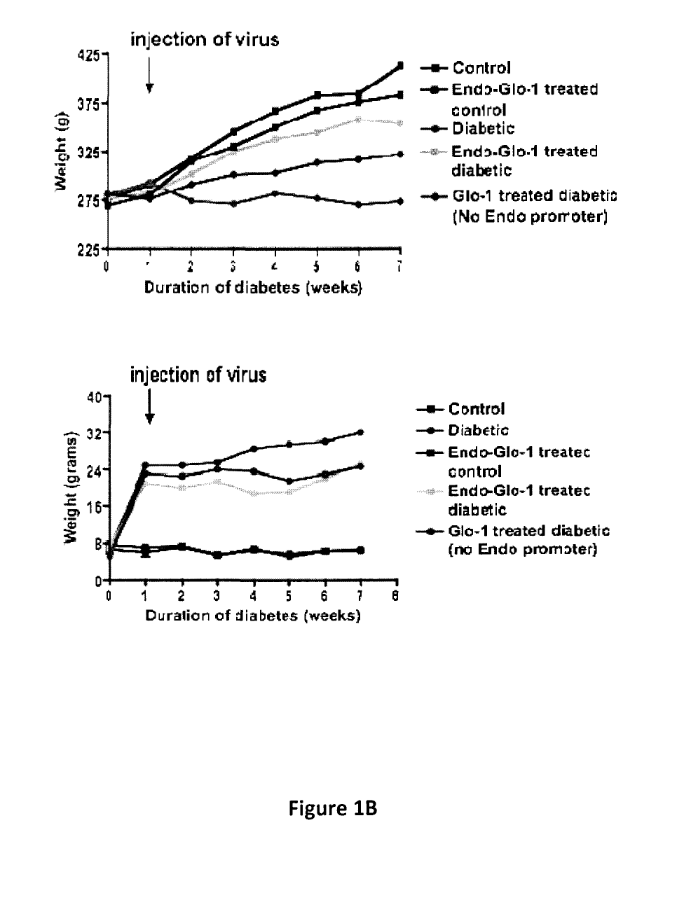

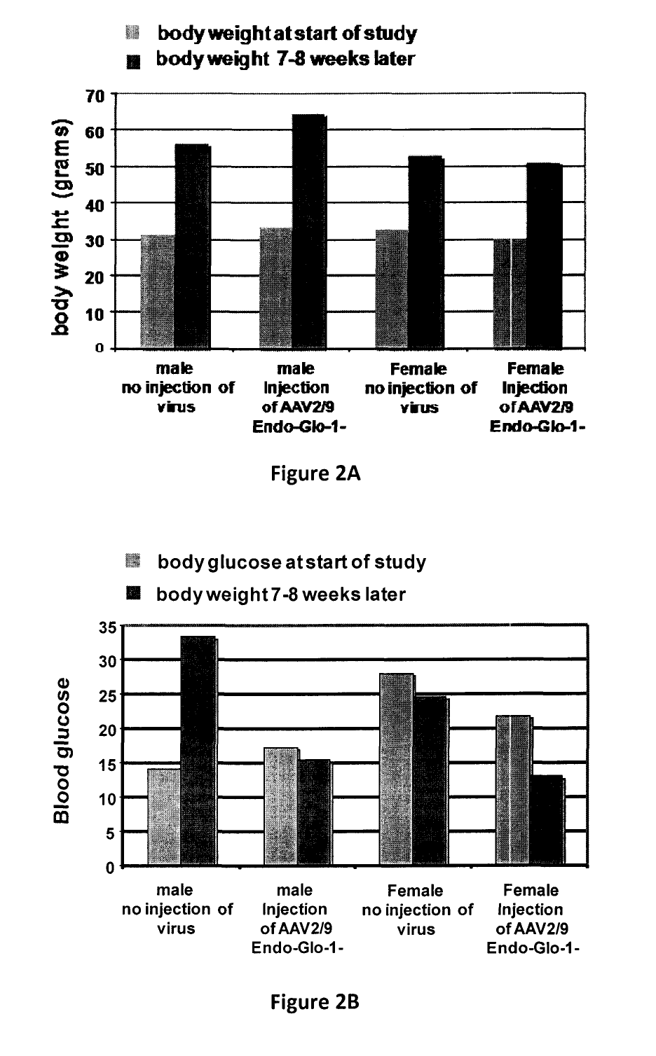

For this study two groups of animals were used. One group had a starting body weight of about 220 g and the other group had a body mass of about 275 g. As seen in FIG. 1, AAV2/9-Endo Glo-1 prevented the body weight loss and lower blood glucose in streptozotocin-induced type 1 diabetic rats for both the ˜220 g group (FIG. 1A) and the ˜275 g group (FIG. 1B). Notably, AAV2/9-EndoGlo-1 injection increased body weight in male but not female db/db type 2 diabetic mice (leptin receptor defective) (FIG. 2A). However, AAV2/9-EndoGlo-1 injection reduced blood glucose in male and female db/db type 2 diabetic mice (leptin receptor defective) (FIG. 2B).

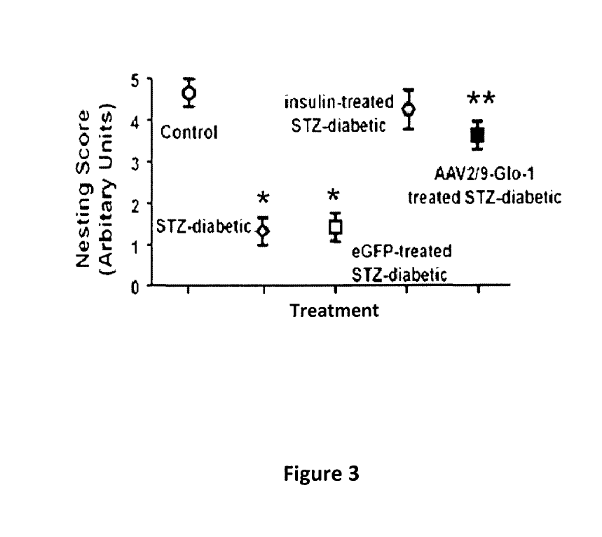

Nest construction may be used to assess daily living functions in both male and female rats (Deacon, R. (2012) J. Vis. Exper., 59:e2607; Deacon et al. (2008) Behavioural Brain Res., 189:126-138; Sager et al. (2010) Behavioural Brain Res., 208:444-449). This test simultaneously assesses attention, decision-making, motor function, and visuo-construction, which requires central, orofacial, and forelimb movements. For this assay, a piece of cotton is placed in one corner of the cage 2 hours prior to dark cycle. After 16 hours, the ability of the rodent to shred the cotton and construct a nest in the cage is assessed. A scale of 1-5 was developed to rank performance, with 5 being complete cotton shredding and nest construction in the center of the cage, and 1 being no shredding or movement of cotton. Using this assay, it was discovered that a single injection of AAV2/9-Glo-1 (8×10¹² viral particles/kg, 200 μl volume) one week after the onset of diabetes improved nest construction in 7-8 weeks T1D male rats (FIG. 3). AAV2/9-Glo-1 administration also reduced the characteristic polyuria (wet cage) in T1D rats and showed some reductions in blood glucose levels. Daily insulin injections to achieve a euglycemic state, starting 5-6 weeks after the onset of T1D, also improved nest construction, establishing that this defect resulted from hyperglycemia (diabetes) and not the diabetogenic agent, streptozotocin.

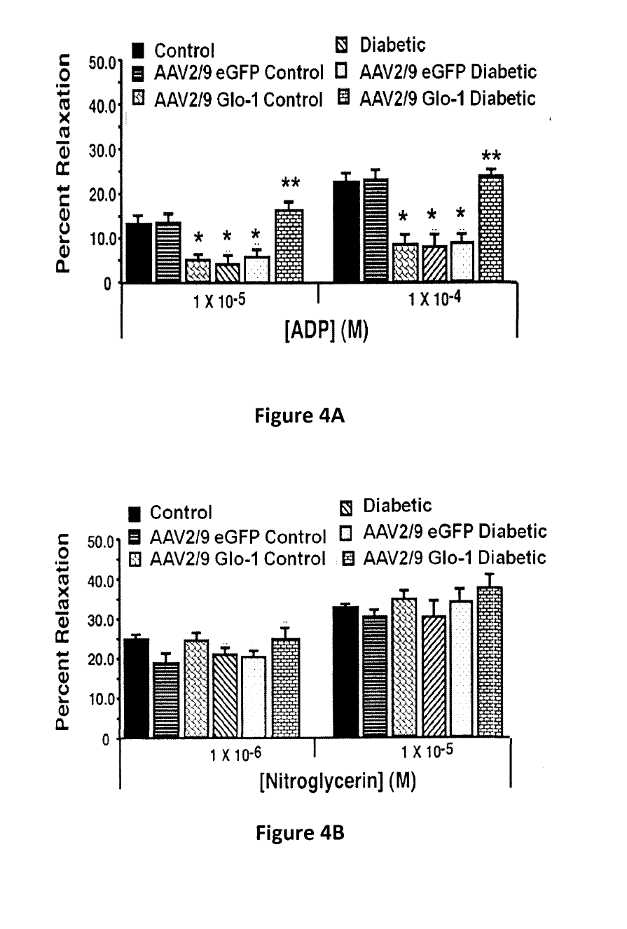

In order to assess vascular reactivity, male rats were intravenously injected with either citrate buffer (pH 4.5, 50 μl volume) or STZ in citrate buffer (45-50 mg/kg, pH 4.5). One week later, control and diabetic rats were subdivided into three groups and injected with either AAV2/9-eGFP (a control virus, 8×10¹² viral particles/kg, 200 μl volume), AAV2/9-Glo-1, or phosphate buffer saline (PBS). Seven to eight weeks later, a craniectomy was performed over the left parietal cortex to visualize the microcirculation of the cerebrum. The cranial window was suffused with artificial cerebrospinal fluid bubbled with 95% nitrogen and 5% carbon dioxide. Video imaging was used to assess changes in the diameters of pial arteriole, in response to the EC nitric oxide synthase (eNOS)-activating ligand adenosine diphosphate (ADP) (10⁻⁴ and 10⁻⁵ M) and eNOS-independent ligand sodium nitroglycerin (10⁻⁶ and 10⁻⁵ M) (Arrick et al. (2011) Amer. J. Physiol. Heart Circul. Physiol., 301:H696-703). Data shows that injection of AAV2/9-Glo-1 improved pial arteriole responsiveness to ADP in T1D rats (FIG. 4). AAV2/9-Glo-1 also prevented diabetes-induced loss of n-NOS-dependent vasodilatation of pial arterioles. Furthermore, AAV2/9-Glo-1 injection in control animals impaired the response of pial arteriole to ADP, attesting to the importance of MG in normal physiology. Consistent with other studies, the responsiveness of the pial arteriole to nitroglycerin was not compromised in T1D (Mayhan, W. G. (1992) Brain Res., 580:297-302) and AAV2/9-Glo-1 injection had no effect on SMC-mediated dilation.

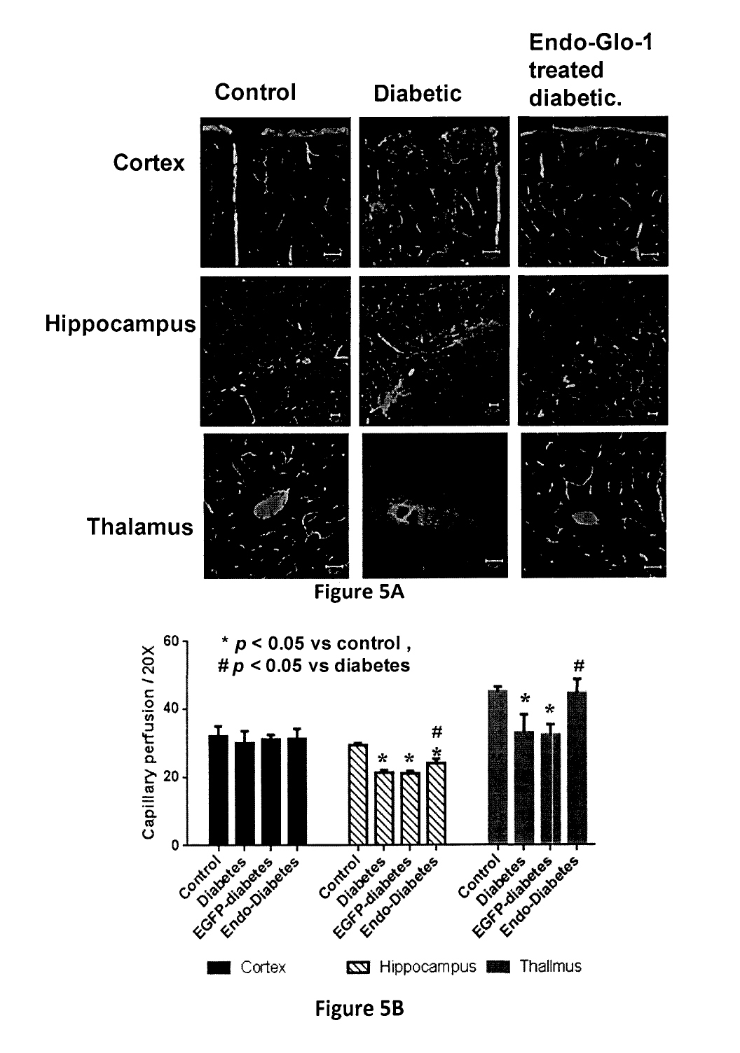

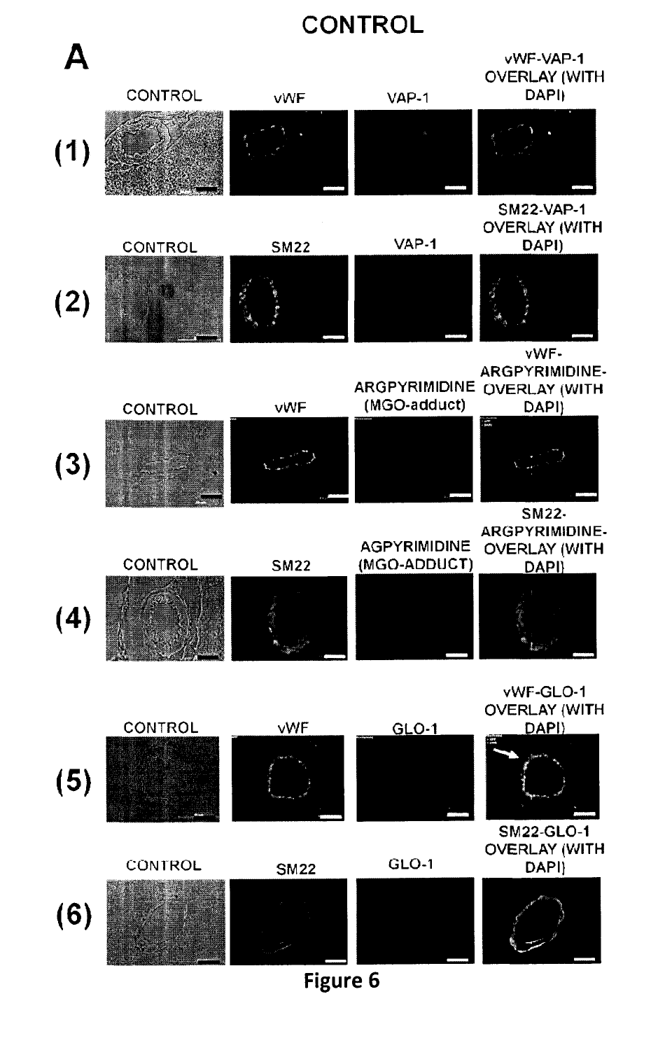

Disruption of the endothelium compromises the permeability of the blood-brain barrier. To assess cerebral vascular leakage, a fluorescent dye composed of bovine serum albumin (BSA) labeled with fluorescein isothiocyanate (BSA-FITC) was used (Lominadze et al. (2006) Amer. J. Phys.: Heart Circ. Phys., 290:H1206-1213). At 15 minutes prior to sacrifice, control, diabetic, and AAV2/9-Glo-1-treated diabetic rats (7-8 weeks) were injected intravenously with BSA-FITC (100 μl, 50 mg/ml). After this time, brains were excised and post-fixed, then sectioned into 30 μm thick coronal slices. Confocal microscopy was used to assess BSA-FITC labeling as an index of cerebral leakage. Immunohistochemistry (IHC) was also conducted to co-localize immunoglobulins (IgG) in areas of leakage and for the glial fibrillary acidic protein (GFAP) to assess the status of astrocytes. In these studies, minimal cerebral leakage was detected in brains of control rats. However, in T1D rats, localized cerebral leakage was observed in several regions of the brain including the cortex, hippocampus, thalamus, and cerebellum. In regions where leakage occurred, staining for IgG was prominent, confirming vascular leakage. In contrast, AAV2/9-Endo Glo-1 treatment prevented cerebral microvascular leakage in 8 week STZ-diabetic rats (FIG. 5A). AAV2/9 Endo-Glo-1 injection also prevented the loss of perfused capillary density in brains of rats with type 1 diabetes (FIG. 5B).

Astrocytes adjacent to vascular leakage site were also “star-shaped,” indicating activation (an index of inflammation). Injection of AAV2/9-Glo-1 one week after the onset of diabetes significantly attenuated cerebral vascular leakage and the presence of star-shaped astrocytes (Kim et al. (2012) Graefe's Arch. Clin. Exper. Ophthalmol., 250:691-697). Indeed, AAV2/9 Endo-Glo-1 injection prevented activation of astrocytes in cortex and thalamus of rats with type 1 diabetes (STZ-induced).

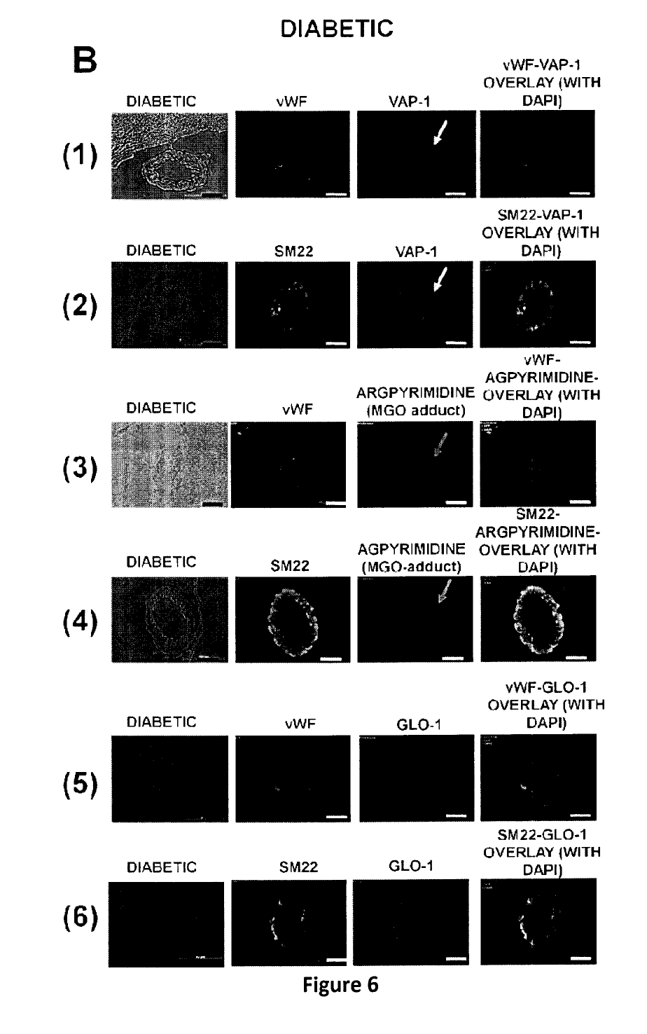

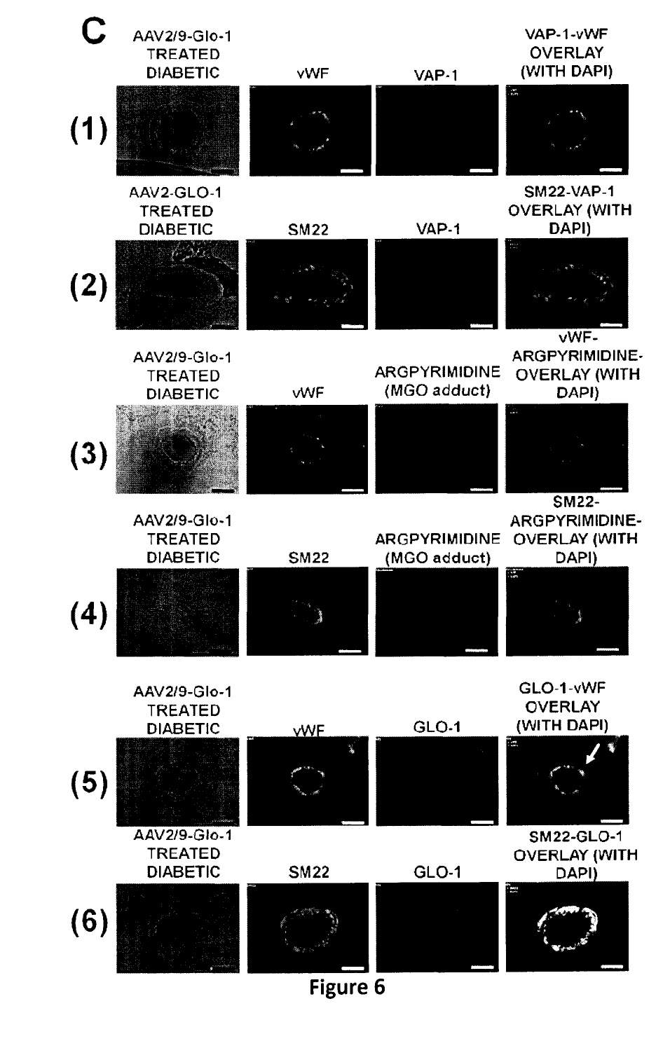

Immunohistochemistry (IHC) studies were conducted to gain insight into how AAV2/9-Glo-1 improves the function of endothelial cells (EC) and, subsequently, cognition. Results are presented in FIG. 6. In these studies, cells were stained for the von Willerbrand factor (vWF), a marker for EC. In cerebral microvessels of T1D rats, vWF staining markers were significantly reduced. Interestingly, there were no significant changes in SM22 staining, a marker for smooth muscle cells (SMC). Concomitant with the reduced vWF staining in diabetic microvessels were significant increases in staining for MG-synthesizing enzyme vascular adhesion protein-1 (VAP-1) and argpyrimidine, a marker for MG in SMC. Expression of Glo-1 was not significantly different from control. A single injection of AAV2/9-Glo-1 one week after the onset of T1D significantly increased expression of Glo-1 in SMC, restored the endothelium, and reduced expression of VAP-1 and argpyrimidine adduct in SMC. Intravenous injection of AAV2/9-Glo-1 did not alter expression of Glo-1 in neurons or astrocytes. Because AAV2/9-Glo-1 did not infect and/or express Glo-1 in EC, it can be concluded that the protection afforded to the EC arose from reduced MG levels in SMC and not from self-protection of EC.

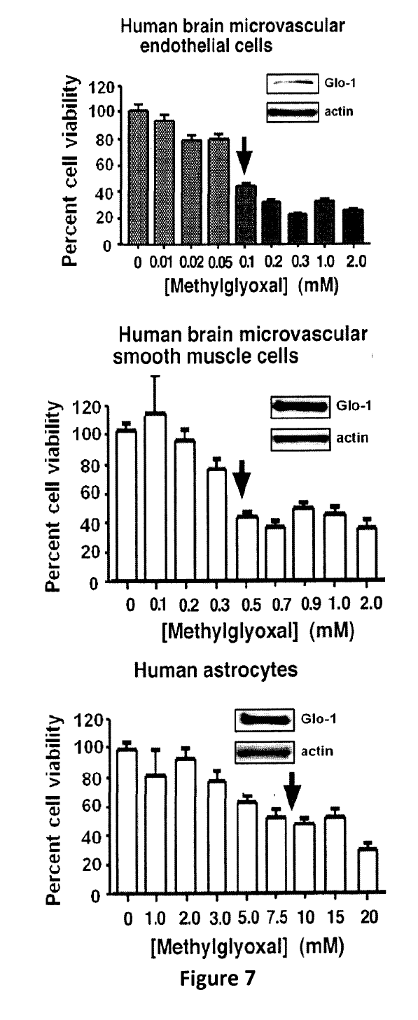

Next, the effect of MG on viability of EC, SMC, and astrocytes was evaluated. For this, human EC, SMC, and astrocytes (˜70% confluency) were incubated for 16 hours with varying amounts of MG, after which cell viabilities were assessed using an MTT assay (Cookson et al. (1995) Toxicol. in vitro, 9:39-48). Data show that MG was more than 5× more toxic to EC than to SMC, and 20× more toxic to EC than astrocytes (FIG. 7). MG reduced the viability of rat cortical neurons with an LD₅₀ similar to that to EC (100 μM). The enhanced susceptibility of human EC and rat cortical neurons to MG is likely due in part to their low steady-state level of MG degradation, Glo-1 (Western blots of FIG. 7). Time-lapsed confocal imaging with appropriate dyes were then used to assess the ability of methylglyoxal to alter cytoplasmic (Fluo-3) and mitochondrial (Rhod-2) Ca²⁺, and ROS production in the mitochondria (MitoSox and Mitotracker green). To confirm ROS generation in mitochondria, mitochondria were purified and electron paramagnetic resonance (EPR) spectroscopy was performed to assess the effect of methylglyoxal on ROS production with mitochondria using the spin probe 1-hydroxy-3-methoxycarbonyl-2,2,5,5-tetramethylpyrrolidine (CMH). Finally, ECs were incubated with varying methylglyoxal and Western blot assays were conducted to assess steady state levels tight junction proteins. These studies revealed that methylglyoxal sequentially increased cytoplasmic and mitochondria Ca²⁺, mitochondrial superoxide (O₂.) and reduced expression of tight junction proteins (e.g., claudin 5 and occludin).

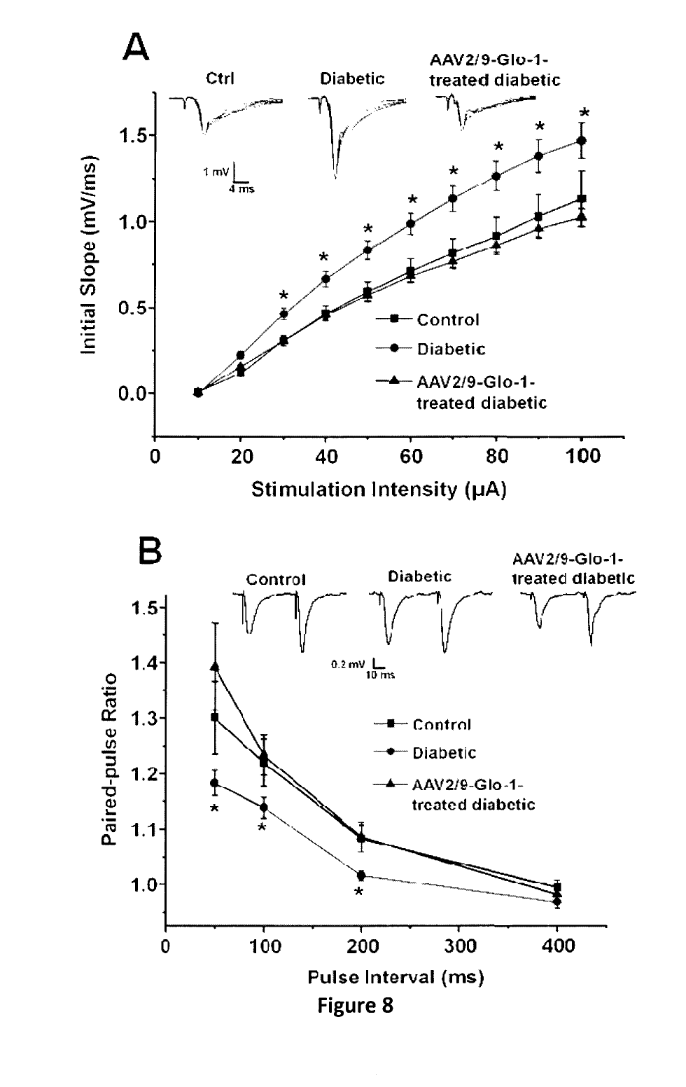

The synaptic transmission in hippocampal slices was assessed using a modification of a previously described procedure (Xiong et al. (1999) AIDS Res. Hum. Retrovir., 15:57-63). Briefly, after anesthesia with pentobarbital-sodium, rats were perfused intracardially with artificial cerebrospinal fluid (ACSF), consisting of 124 mM sodium chloride (NaCl), 3 mM potassium chloride (KCl), 2 mM calcium chloride (CaCl₂), 2 mM magnesium chloride (MgCl₂), 1 mM sodium dihygrogen phosphite (NaH₂PO₃), 26 mM sodium carbonate (NaCO₃), and 10 mM Glucose (95% O₂, and 5% CO₂, pH of 7.3-7.5) for 10 minutes. Brains were then quickly removed and placed in ice-cold (4° C.), oxygenated ACSF. Hippocampi were removed and sectioned into 400 μm transverse slices, which were stabilized by oxygenation in ACSF for 1 hour. Field excitatory postsynaptic potentials (fEPSPs) were then elicited by constant-current, low-frequency orthodromic stimulation (0.05 Hz) of Schaffer-collateral-commissural axons using an insulated, bipolar, tungsten electrode. Evoked fEPSPs were recorded in the CA1-dendrite field (stratum radiatum) to determine the slope of the input-output (IO) response. The stimulus duration for the current was fixed at 40 μs; the stimulus intensity varied from 10 μA to 100 μA, at increments of 10 μA. Also, paired pulse facilitation (PPF) curves were generated by testing the Schaffer-collateral pathway with twin pulses at 40 μsec intervals, with interpulse intervals ranging from 50-400 msec. The paired pulses were delivered at 20-sec intervals, and six consecutive responses were averaged for each. The degree of facilitation was determined by the increase in the ratio between the amplitude of the second response over the first response. Data show that the initial slopes and amplitudes of IO curves from T1D hippocampi slices were greater than those of controls (FIG. 8). The PPF at lower pulse intervals also decreased in T1D animals consistent with impaired pre-synaptic function. A single injection of AAv2/9-Glo-1 one week after the onset of T1D blunted changes in synaptic transmission (FIG. 8). These data establish that AAv2/9-Glo-1 prevents neuronal dysregulation in T1D rats.

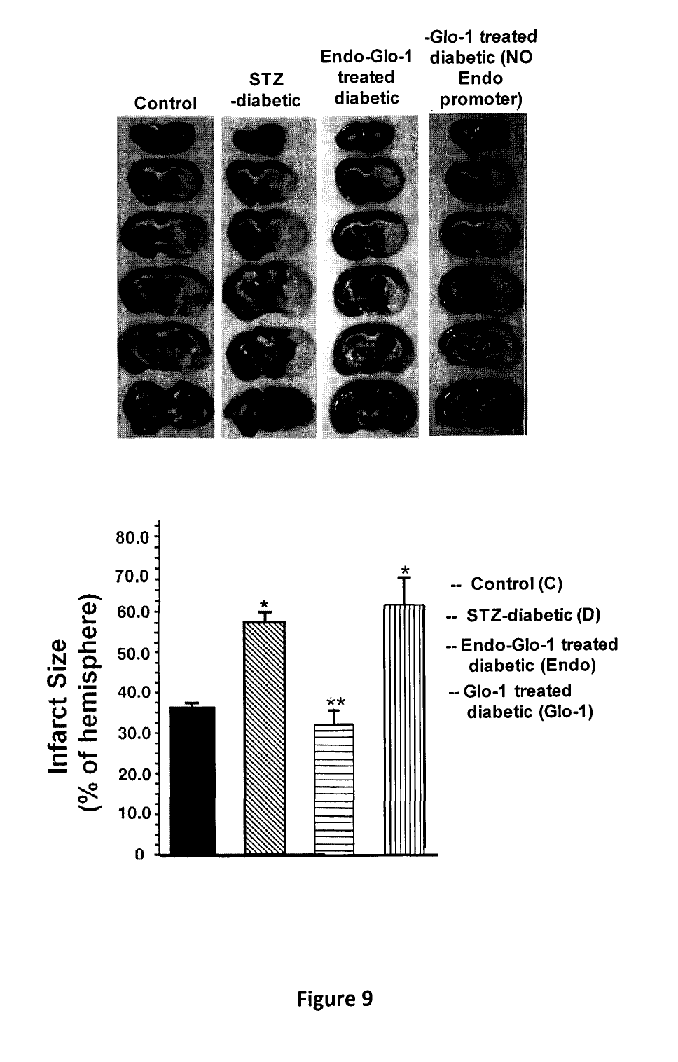

To assess if AAV2/9-Glo-1 protected against ischemia-reperfusion injury, another major cerebral co-morbidity in T1D (Bruno et al. (2010) Current Treatment Options Neurol., 12:492-503), rats were anesthetized with ketamine/xylazine. The right femoral artery was cannulated for continuous monitoring of mean arterial blood pressure (MABP). Rectal temperatures were maintained at 37° C. using a temperature-controlled heating pad. A laser Doppler flow probe attached to the right side of the dorsal surface of the skull 1-2 mm caudal and 5-6 mm lateral to bregma was used to measure regional cerebral blood flow (rCBF). The right common and external carotid arteries of each rat were exposed. A mid-cerebral artery (MCA) was occluded using intraluminal suture occlusion. The suture was carefully withdrawn 1.5 hours after occlusion. The incisions were sutured, and animals were allowed to recover for 24 hours. Rats were anesthetized with thiobutabarbital sodium and exsanguinated. The brains were quickly removed and placed on ice-cold sterile saline for 5 min and cut into six 2-mm coronal sections. Sections were stained with 2% 2,3,5-triphenyltetrazolium chloride (TTC). Slice images were digitalized, and the ischemic lesions were evaluated using Kodak Molecular Imaging Software. The total and infarct lesions, which were corrected for cerebral edema, were expressed as percentage of the contra-lateral hemisphere (Zhao et al. (2008) Brain Res., 1246:158-166). Data show that the infarct volume was greater in T1D rats compared to controls (FIG. 9). Single AAV2/9-Glo-1 injection one week after the induction of diabetes prevented the exaggerated infarct following ischemia-reperfusion (FIG. 9). When AAV2/9-Glo-1 without the endothelial promoter (AAV2/9-Glo-1 (No Endo-1)) was injected one week after diabetes, AAV2/9-Glo-1 (No Endo-1) did not prevent ischemia-perfusion injury, emphasizing the uniqueness of AAV2/9-Glo-1 driven by the endothelin-promoter.

If the increase in MG is an underlying cause for heart failure in DM, lowering MG level should blunt heart failure development in diabetes. Two adeno-associated viruses were created to test this hypothesis. One virus was designed to simulate global overexpression of Glo-1 (AAV2/9-Glo-1). The other was driven by the endothelin-1 promoter, (AAV2/9-Endo-Glo1) to express Glo-1 expression in smooth muscle cells. Viruses were injected via a tongue vein in rats (200 μL of 1×10¹² pfu/kg) one week after injection STZ in rats. After 5-6 weeks, cardiac function was assessed using M-mode echocardiography. Animals were then sacrificed, and myocytes were isolated and used to assess Ca²⁺ transients. The results are provided in Table 1. Surprisingly, % fractional shortening and ejection fraction in both AAV2/9-Glo-1 and AAV2/9-Endo-1-Glo-1 treated T1D rats were significantly better than those of T1D rats. As before, mean body weight of AAV-Glo-1- and AAV-Endo-Glo-1-treated diabetic rats were significantly higher than that of STZ-diabetic animals, p<0.05. Additionally, it was determined that AAV-Endo-Glo1 treated diabetic animals also had significantly lower glucose levels over the study period.

TABLE 1
Cardiac function.

| | | | AAV2/9-Glo- | AAV2/9- | |
|---|---|---|---|---|---|
| | eGFP- | eGFP- | 1 treated | Endo-1-Glo- | STZ-diabetic |
| | treated | treated STZ- | STZ- | 1 treated | treated with |
| | control | diabetic | diabetic | STZ-diabetic | insulin |
| Parameter | (n = 8) | (n = 9) | (n = 6) | (n = 6) | (n = 5) |
| Body weight | 380.3 ± 6.1 | 277.1 ± 8.2* | 320.1 ± 10.2** | 359.1 ± 7.1** | 320.6 ± 7.2** |
| (g) | | | | | |
| Blood | 4.5 ± 0.5 | 28.1 ± 3.3* | 23.8 ± 4.1 | 19.2 ± 2.1** | 20.2 ± 2.3** |
| glucose | | | | | |
| (mmol/L) | | | | | |
| Serum | 1.5 ± 0.2 | 0.4 ± 0.1* | 0.6 ± 0.1 | 0.6 ± 0.1 | 1.0 ± 0.2** |
| insulin | | | | | |
| (μg/ml) | | | | | |
| Heart rate | 367.1 ± 10.1 | 280.8 ± 15.1 | 307.2 ± 8.1 | 329.2 ± 5.1* | 300.6 ± 6.1 |
| (bpm) | | | | | |
| % fractional | 49.1 ± 1.7 | 41.7 ± 1.9* | 47.5 ± 0.7** | 50.2 ± 1.6** | 42.1 ± 1.9* |
| shortening | | | | | |
| Ejection | 79.1 ± 1.9 | 70.9 ± 1.8* | 77.4 ± 0.7** | 80.1 ± 1.6** | 71.2 ± 1.8* |
| fraction | | | | | |

Cardiac function in diabetic rats treated with insulin to achieve the same glycemic level as T1D treated with AAV-Endo-Glo-1, was significantly lower than those in T1D treated with AAV-Endo-Glo-1. These data indicate that improvement in metabolic milieu by AAV2/9-Endo-Glo-1 was insufficient account for the improvement in cardiac function. Evoked Ca²⁺ transient kinetics also improved significantly in myocytes from AAV-Endo-Glo1 and AAV-Glo-1-treated diabetic animals. Western blots confirmed AAV-Glo-1 and AAV-Endo-Glo-1 increased expression of Glo-1 in smooth muscle cells.

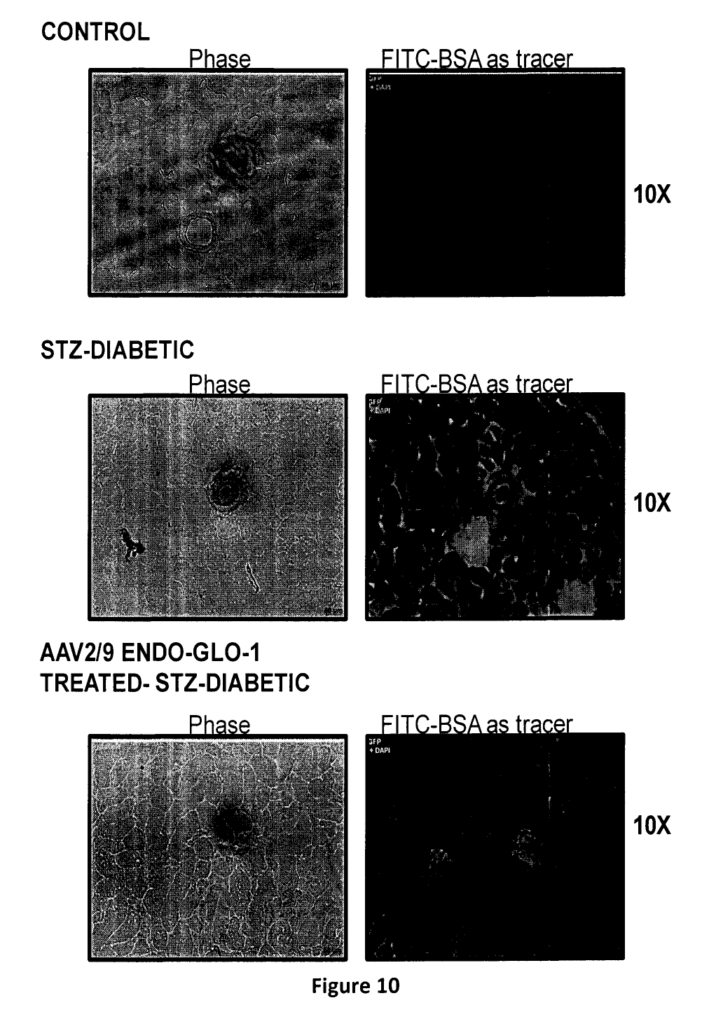

It was also determined if AAV2/9-Endo-Glo-1 was able to blunt vascular/glomeruli leakage in the kidneys of streptozotocin (STZ)-induced diabetic rats. For this, rats were injected with either citrate buffer (control) or streptozotocin (45 mg/kg, iv). Seven days after STZ injection, diabetic rats were separated into two groups; one group of diabetic rats were given a single injection of AAV2/9-Endo-Glo-1 (8×10¹² viral particles/kg, i.v., 200 μl volume), while the other group continued as diabetic. Seven to eight weeks later, animals were anesthetized and bovine serum albumin labeled with fluorescein isothiocyanate (BSA-FITC, 200 μl of 50 mg/ml) was injected and allowed to circulate for 15 minutes. After this time animals were sacrificed, kidneys were excised and fixed 4% paraformaldehyde cut into 30 μm thick sections and cortex were analyzed for vascular leakage and “tubule function” using confocal microscopy. FIG. 10 shows low magnification (10×) of kidney sections from control, STZ-diabetic and AAV2/9-Endo-Glo-1 treated STZ diabetic animals. In control animals, BSA-FITC was confined to the lumen of the tubules and there was no leakage of BSA-FITC from the blood vessels or in glomeruli (arrow). Kidneys from STZ-diabetic rats show extensive BSA-FITC around the walls of the tubules, leakage from blood vessels. Glomeruli from STZ-diabetic kidneys also contained a significant amount of BSA-FITC dye. Administration of AAV2/9-Endo-Glo-1 to one week after the onset of diabetes significantly reduced leakage of BSA-FITC from the blood vessels and within glomeruli. BSA-FITC was also confined to the lumen of the tubules. It should be re-emphasized that no agents were used to lower blood glucose in STZ-diabetic animals.

If the increase in MG is an underlying cause for heart failure in DM, lowering MG level should blunt heart failure development in mice with type 2 diabetes. Two adeno-associated viruses were created to test this hypothesis. AAV2/9-Endo-Glo1 was injected in male and female db/db mice via a tongue vein (200 μL of 1×10¹² pfu/kg) at 3 months of age. After 7-8 weeks, cardiac function was assessed using M-mode echocardiography. The first finding was that % fractional shortening and ejection fraction in male mice injected with AAV2/9-Endo-1-Glo-1 were significantly lower than in male db/db mice not injected with the virus. Interestingly male mice injected with AAV2/9-Endo-1-Glo-1 had significantly lower blood glucose than male nice not injected with the virus.

The second finding was that % fractional shortening and ejection fraction in female mice injected with AAV2/9-Endo-1-Glo-1 were significantly higher than in female db/db mice not injected with the virus. Female mice injected with AAV2/9-Endo-1-Glo-1 also had significantly lower blood glucose than female mice not injected with the virus.

TABLE 2
Type 2 diabetic mice.

| | | db/db male | | db/db female |
|---|---|---|---|---|
| | | Injected with | | Injected with |
| | db/db male | AAV2/9- | db/db female | AAV2/9- |
| | no injection | Endo-Glo-1 | no injection | Endo-Glo-1 |
| Parameter | (n = 4) | (n = 4) | (n = 4) | (n = 4) |
| Body weight | 50.5 ± 0.1 | 48.4 ± 3.0 | 48.4 ± 3.0 | 51.1 ± 2.4 |
| (grams) | | | | |
| Blood glucose | 33.2 ± 2.1 | 15.0 ± 2.1* | 24.9 ± 2. | 13.8 ± 2.1* |
| (mmol/L) | | | | |
| % fractioning | 41.9 ± 0.8 | 32.6 ± 3.1* | 32.8 ± 0.6 | 37.6 ± 0.5** |
| shortening | | | | |
| Ejection | 74.0 ± 1.0 | 62.1 ± 5.7* | 62.4 ± 1.0 | 69.4 ± 0.9** |
| fraction | | | | |

*denotes significant reduction from untreated, p < 0.05
**denotes significant increase from untreated, p < 0.05

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for inhibiting a disease or disorder characterized by overexpression of methylglyoxal in a subject, said method comprising administering to said subject a nucleic acid molecule encoding a methylglyoxal degrading enzyme.

2. The method of claim 1, wherein said methylglyoxal degrading enzyme is glyoxalase-1.

3. The method of claim 1, wherein said disease or disorder characterized by overexpression of methylglyoxal is diabetes or diabetes related complications.

4. The method of claim 1, wherein said disease or disorder characterized by overexpression of methylglyoxal is vascular disease.

5. The method of claim 3, wherein said disease or disorder characterized by overexpression of methylglyoxal is a diabetes related complication selected from the group consisting of organ dysfunction, vascular disease, cardiovascular disease, heart failure, arterial atherogenesis, renal failure, retinopathy, neuropathy and cognitive impairment.

6. The method of claim 5, wherein said diabetes related complication is cognitive impairment.

7. The method of claim 5, wherein said diabetes related complication is cardiovascular disease.

8. The method of claim 1, wherein said glyoxalase-1 is human glyoxalase-1.

9. The method of claim 1, wherein said method comprises administering to said subject a vector comprising said nucleic acid molecule encoding a methylglyoxal degrading enzyme.

10. The method of claim 9, wherein said nucleic acid molecule encoding a methylglyoxal degrading enzyme is operably linked to an endothelial cell promoter.

11. The method of claim 10, wherein said endothelial cell promoter is the endothelin-1 promoter.

12. The method of claim 9, wherein said nucleic acid molecule encoding a methylglyoxal degrading enzyme is operably linked to an smooth muscle cell promoter.

13. The method of claim 9, wherein said vector is a viral vector.

14. The method of claim 13, wherein said viral vector is an adeno-associated viral vector.

15. The method of claim 14, wherein said adeno-associated viral vector comprises a capsid protein of serotype 9.

16. The method of claim 15, wherein said nucleic acid molecule encoding a methylglyoxal degrading enzyme is operably linked to the endothelin-1 promoter.

17. The method of claim 16, wherein said methylglyoxal degrading enzyme is glyoxalase-1.

18. A composition comprising a vector comprising a nucleic acid molecule encoding a methylglyoxal degrading enzyme and a pharmaceutically acceptable carrier, wherein said nucleic acid molecule encoding a methylglyoxal degrading enzyme is operably linked to an endothelial cell promoter or a smooth muscle cell promoter.

19. The composition of claim 18, wherein said vector is an adeno-associated viral vector.

20. The composition of claim 19, wherein said adeno-associated viral vector comprises a capsid protein of serotype 9.

21. The composition of claim 20, wherein said nucleic acid molecule encoding methylglyoxal degrading enzyme is operably linked to the endothelin-1 promoter.

22. The composition of claim 21, wherein said methylglyoxal degrading enzyme is glyoxalase-1.
